(19) United States
(12) Patent Application Publication
(10) Pub. No.: US 2015/0342481 A1
(43) Pub. Date: Dec. 3, 2015

(54) MOTION COMPENSATION FOR OPTICAL HEART RATE SENSORS

(71) Applicant: Microsoft Corporation, Redmond, WA (US)

(72) Inventors: Zongyi Liu, Issaquah, WA (US); Haithem Albadawi, Redmond, WA (US)

(21) Appl. No.: 14/292,570

(22) Filed: May 30, 2014

(51) Int. Cl. A61B 5/024; A61B 5/00

(52) U.S. Cl. CPC A61B 5/02427; A61B 5/721

(57) ABSTRACT

A method for an optical heart rate sensor comprises recognizing a minimum amount of motion of the optical heart rate sensor during a testing duration and recognizing an average amount of motion of the optical heart rate sensor during the testing duration. The motion of the optical heart rate sensor is compensated for if the minimum amount of motion during the testing duration exceeds a first threshold, even if the average amount of motion during the testing duration is less than a second threshold, greater than the first threshold. The motion of the optical heart rate sensor is not compensated for if the minimum amount of motion during the testing duration is below the first threshold, even if the average amount of motion during the testing duration is greater than the second threshold.

### BRIEF DESCRIPTION OF THE DRAWINGS

FIGS. 1A and 1B show a wearable electronic device.

FIG. 2 schematically shows an example optical heart rate sensor that may be included in the wearable electronic device of FIGS. 1A-1B.

FIGS. 3A-3C show depictions of a user wearing a sensory-and-logic system and example data traces output by the sensory-and-logic system.

FIG. 4 shows an example method for determining whether to apply motion compensation to an optical signal.

FIG. 5 shows an example data trace output by a motion sensor before and after processing.

FIG. 6 schematically shows a sensory-and-logic system usable to apply motion compensation to an optical signal during predetermined conditions.

### DETAILED DESCRIPTION

The present disclosure is directed to motion compensation for an optical heart rate sensor. For example, motion compensation may be applied to an optical signal when the minimum amount of motion of the optical heart rate sensor exceeds a threshold over a testing duration. While described below in the context of a wearable computing device, it is to be understood that applying motion compensation to an optical signal for an optical heart rate sensor, as described herein, may be used in numerous different applications, and with various different types of sensory-and-logic systems.

Incorporating an optical heart rate sensor into a wearable computing device allows a user to monitor health factors, such as heart rate, calories burned, response to exertion exercises, heart rate variability, etc. However, the signal from the optical sensor may degrade in quality with increased motion, as user motion may change the optical properties of the skin, tissues, and blood vessels beneath the optical sensor. As such, motion compensation may be applied to an optical signal prior to discerning a heart rate from the optical signal. However, motion compensation must be applied selectively. If applied too conservatively, the motion frequencies in the optical signal may be mistaken for heart beats. If applied to aggressively, the heart rate frequencies in the optical signal may be filtered out, leaving only random noise.

The application of motion compensation is particularly challenging during so-called “hand-hold exercises”, such as when a user is riding a stationary bicycle. If the wearable computing device is a wrist-worn form factor, the computing device may realize relatively low amounts of movement while the user's hand is anchored to a handle bar of the stationary bicycle. However, the continuous movement of the user's lower body, in particular the user's footfalls, affect the signal output by the optical sensor, independent of the magnitude of motion indicated by a motion sensor coupled to the wearable computing device. Methods which determine whether to apply motion compensation based on a magnitude of movement may fail to apply motion compensation during hand-hold workouts, and thus indicate inaccurate heart rates. If the movement threshold is set relatively low, sporadic movement while the user is at rest may trigger motion compensation when not warranted.

According to this disclosure, a minimum amount of motion over a testing duration may be used to determine whether to apply motion canceling. Thus, in the case of a hand-hold exercise, the constant, low intensity movement of the user's hand will be enough to trigger motion compensation. However, while at rest, sporadic movements will not affect the minimum amount of motion of the user, and thus motion compensation will not be applied.

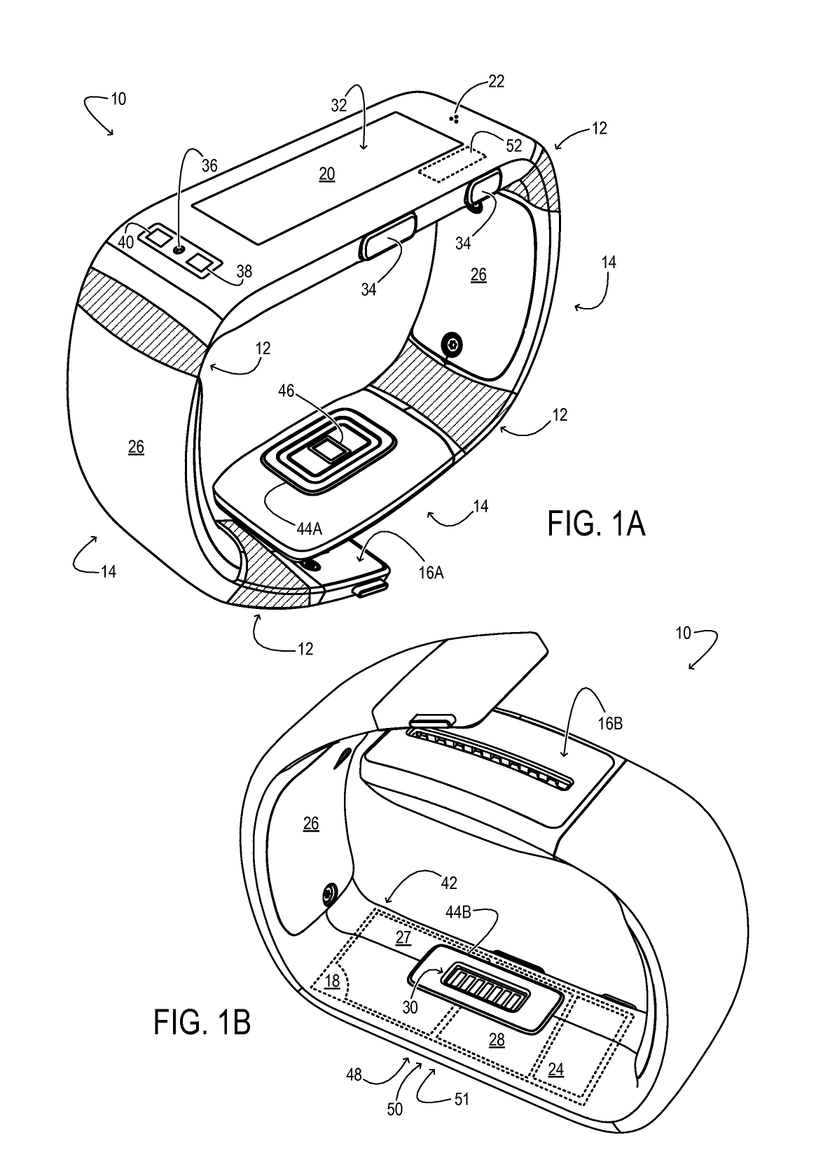

FIGS. 1A and 1B show aspects of an example sensory-and-logic system in the form of a wearable electronic device 10. The illustrated device is band-shaped and may be worn around a wrist. Device 10 includes at least four flexion regions 12 linking less flexible regions 14. The flexion regions of device 10 may be elastomeric in some examples. Fastening componentry 16A and 16B is arranged at both ends of the device. The flexion regions and fastening componentry enable the device to be closed into a loop and to be worn on a user's wrist. In other implementations, wearable electronic devices of a more elongate band shape may be worn around the user's bicep, waist, chest, ankle, leg, head, or other body part. The device, for example, may take the form of eye glasses, a head band, an arm-band, an ankle band, a chest strap, or an implantable device to be implanted in tissue.

Wearable electronic device 10 includes various functional components integrated into regions 14. In particular, the electronic device includes a compute system 18, display 20, loudspeaker 22, communication suite 24, and various sensors. These components draw power from one or more energy-storage cells 26. A battery—e.g., a lithium ion battery—is one type of energy-storage cell suitable for this purpose. Examples of alternative energy-storage cells include super- and ultra-capacitors. In devices worn on the user's wrist, the energy-storage cells may be curved to fit the wrist, as shown in the drawings.

In general, energy-storage cells 26 may be replaceable and/or rechargeable. In some examples, recharge power may be provided through a universal serial bus (USB) port 30, which includes a magnetic latch to releasably secure a complementary USB connector. In other examples, the energy storage cells may be recharged by wireless inductive or ambient-light charging. In still other examples, the wearable electronic device may include electro-mechanical componentry to recharge the energy storage cells from the user's adventitious or purposeful body motion. For example, batteries or capacitors may be charged via an electromechanical generator integrated into device 10. The generator may be turned by a mechanical armature that turns while the user is moving and wearing device 10.

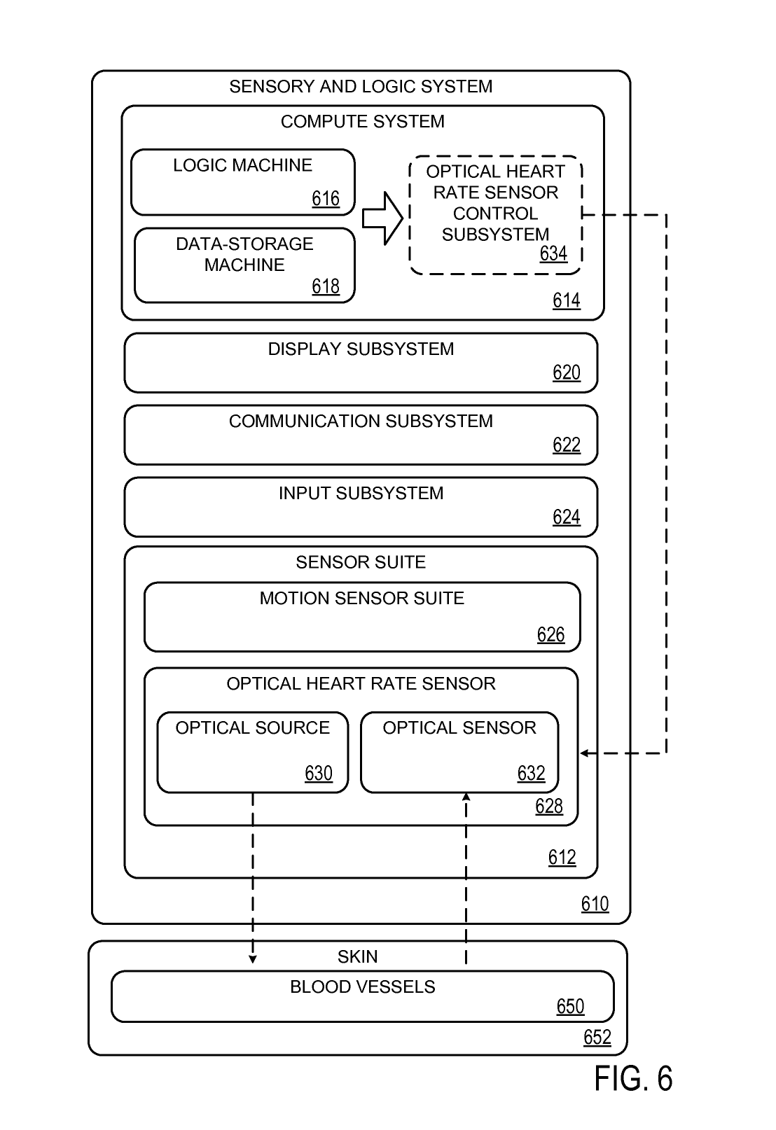

In wearable electronic device 10, compute system 18 is situated below display 20 and operatively coupled to the display, along with loudspeaker 22, communication suite 24, and the various sensors. The compute system includes a data-storage machine 27 to hold data and instructions, and a logic machine 28 to execute the instructions. Aspects of the compute system are described in further detail with reference to FIG. 6.

Display 20 may be any suitable type of display. In some configurations, a thin, low-power light emitting diode (LED) array or a liquid-crystal display (LCD) array may be used. An LCD array may be backlit in some implementations. In other implementations, a reflective LCD array (e.g., a liquid crystal on silicon, LCOS array) may be frontlit via ambient light. A curved display may also be used. Further, AMOLED displays or quantum dot displays may be used.

Communication suite 24 may include any appropriate wired or wireless communications componentry. In FIGS. 1A and 1B, the communications suite includes USB port 30, which may be used for exchanging data between wearable electronic device 10 and other computer systems, as well as providing recharge power. The communication suite may further include two-way Bluetooth, Wi-Fi, cellular, near-field communication and/or other radios. In some implementations, the communication suite may include an additional transceiver for optical, line-of-sight (e.g., infrared) communication.

In wearable electronic device 10, touch-screen sensor 32 is coupled to display 20 and configured to receive touch input from the user. The touch sensor may be resistive, capacitive, or optically based. Pushbutton sensors may be used to detect the state of push buttons 34, which may include rockers. Input from the pushbutton sensors may be used to enact a home-key or on-off feature, control audio volume, turn the microphone on or off, etc.

FIGS. 1A and 1B show various other sensors of wearable electronic device 10. Such sensors include microphone 36, visible-light sensor 38, ultraviolet sensor 40, and ambient temperature sensor 42. The microphone provides input to compute system 18 that may be used to measure the ambient sound level or receive voice commands from the wearer. Input from the visible-light sensor, ultraviolet sensor, and ambient temperature sensor may be used to assess aspects of the wearer's environment—i.e., the temperature, overall lighting level, and whether the wearer is indoors or outdoors.

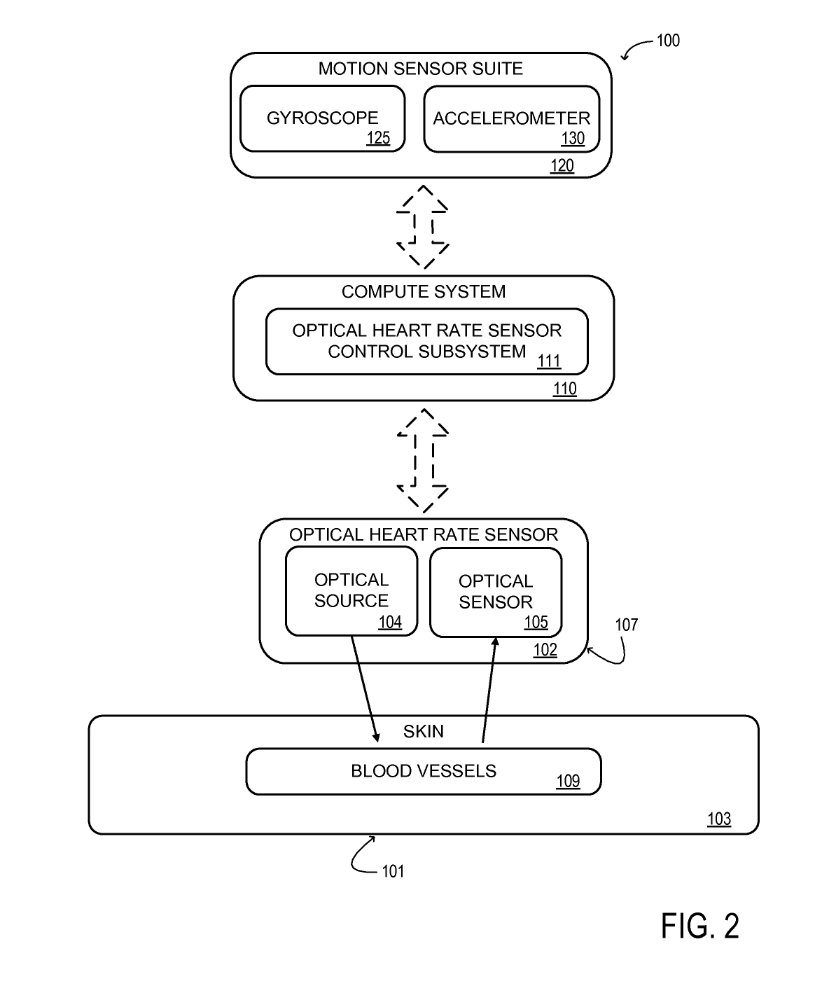

FIGS. 1A and 1B show a pair of contact sensor modules 44A and 44B, which contact the wearer's skin when wearable electronic device 10 is worn. The contact sensor modules may include independent or cooperating sensor elements, to provide a plurality of sensory functions. For example, the contact sensor modules may provide an electrical resistance and/or capacitance sensory function, which measures the electrical resistance and/or capacitance of the wearer's skin. Compute system 18 may use such input to assess whether or not the device is being worn, for instance. In some implementations, the sensory function may be used to determine how tightly the wearable electronic device is being worn. In the illustrated configuration, the separation between the two contact-sensor modules provides a relatively long electrical path length, for more accurate measurement of skin resistance. In some examples, a contact sensor module may also provide measurement of the wearer's skin temperature. Arranged inside contact sensor module 44B in the illustrated configuration is the optical heart rate sensor 46. The optical heart rate sensor may include an optical source and matched optical sensor to detect blood flow through the capillaries in the skin and thereby provide a measurement of the wearer's heart rate, blood oxygen level, blood glucose level, or other biomarkers with optical properties. Further details regarding the optical heart rate sensor, optical source, and optical sensor are provided with reference to FIG. 2.

Wearable electronic device 10 may also include motion sensing componentry, such as an accelerometer 48, gyroscope 50, and magnetometer 51. The accelerometer and gyroscope may furnish inertial and/or rotation rate data along three orthogonal axes as well as rotational data about the three axes, for a combined six degrees of freedom. This sensory data can be used to provide a pedometer/calorie-counting function, for example. Data from the accelerometer and gyroscope may be combined with geomagnetic data from the magnetometer to further define the inertial and rotational data in terms of geographic orientation. The wearable electronic device may also include a global positioning system (GPS) receiver 52 for determining the wearer's geographic location and/or velocity. In some configurations, the antenna of the GPS receiver may be relatively flexible and extend into flexion regions 12.

Compute system 18, via the sensory functions described herein, is configured to acquire various forms of information about the wearer of wearable electronic device 10. Such information must be acquired and used with utmost respect for the wearer's privacy. Accordingly, the sensory functions may be enacted subject to opt-in participation of the wearer. In implementations where personal data is collected on the device and transmitted to a remote system for processing, that data may be anonymized. In other examples, personal data may be confined to the wearable electronic device, and only non-personal, summary data transmitted to the remote system.

FIG. 2 shows a schematic depiction of a sensory-and-logic system 100 coupled to the wrist of a user 101 so that an optical heart rate sensor 102 is adjacent to the skin 103 of user 101. Optical heart rate sensor 102 comprises an optical source 104 configured to illuminate one or more blood vessels through the skin of the user, and an optical sensor 105, configured to measure reflected illumination from the blood vessels. Optical source 104 may comprise one or more LED emitters, for example, while optical sensor 105 may comprise one or more photodiodes matched to detect light at frequencies that are based on the frequencies of light output by the optical source. Optical heart rate sensor 102 may be coupled within a housing 107 configured to promote contact between sensor 102 and skin 103, and further configured to block, filter, or otherwise limit ambient light from reaching the optical sensor. In this way, the majority of light reaching optical sensor 105 may be light originating from optical source 104 that has reflected off of blood vessels 109 beneath skin 103. As an example, FIG. 1A shows a wearable electronic device 10 that is configured to position optical heart rate sensor 46 such that its optical source may illuminate capillaries located beneath the skin of the user's forearm while the wearable electronic device is worn by the user. In other configurations, an optical heart rate sensor may be positioned within a wearable electronic device such that an optical source illuminates a radial artery through the skin of the user while the wearable electronic device is worn by the user. Alternatively, an optical heart rate sensor and its associated compute system may be housed separately and configured to communicate via a communication suite. For example, an optical heart rate sensor may be included in a head set and configured to illuminate capillaries located in the user's ear lobe while the head set is worn by the user, while the compute system resides within a wrist-worn computing device configured to communicate with the head set, via wireless communication, for example. An optical sensor may be configured to sense light reflected off of blood vessels located beneath the skin of the user (e.g., wrist worn), or the optical sensor may be configured to sense light transmitted through blood vessels located beneath the skin of the user (e.g., ear worn).

Compute system 110 may comprise optical heart rate sensor control subsystem 111. Optical heart rate sensor control subsystem 111 may provide control signals to optical source 104 and optical sensor 105. Optical heart rate sensor control subsystem 111 may receive raw signals from optical sensor 105, and may further process the raw signals to determine heart rate, caloric expenditures, etc. Processed signals may be stored and output via compute system 110. Control signals sent to optical source 104 and optical sensor 105 may be based on signals received from optical sensor 105, one or more motion sensors, ambient light sensors, information stored in compute system 110, input signals, etc.

The signal from the optical sensor may degrade in quality with increased motion, as user motion may change the optical properties of the skin, tissues, and blood vessels beneath the optical sensor. Further, user motion may impact the movement of blood and other fluids through the user's tissue. As such, the signal output by the optical sensor may need to be filtered or otherwise adjusted based on user movement prior to determining a heart rate of the user. Sensory-and-logic system 100 may include a motion sensor suite 120 communicatively coupled to compute system 108. Signals from motion sensor suite 120 may be provided to optical heart rate control subsystem 111. Motion sensor suite 120 may include gyroscope 125 and accelerometer 130. Gyroscope 125 and accelerometer 130 may be three-axis motion sensors. Accordingly, gyroscope 125 and accelerometer 130 may record and transmit signal channels for each axis.

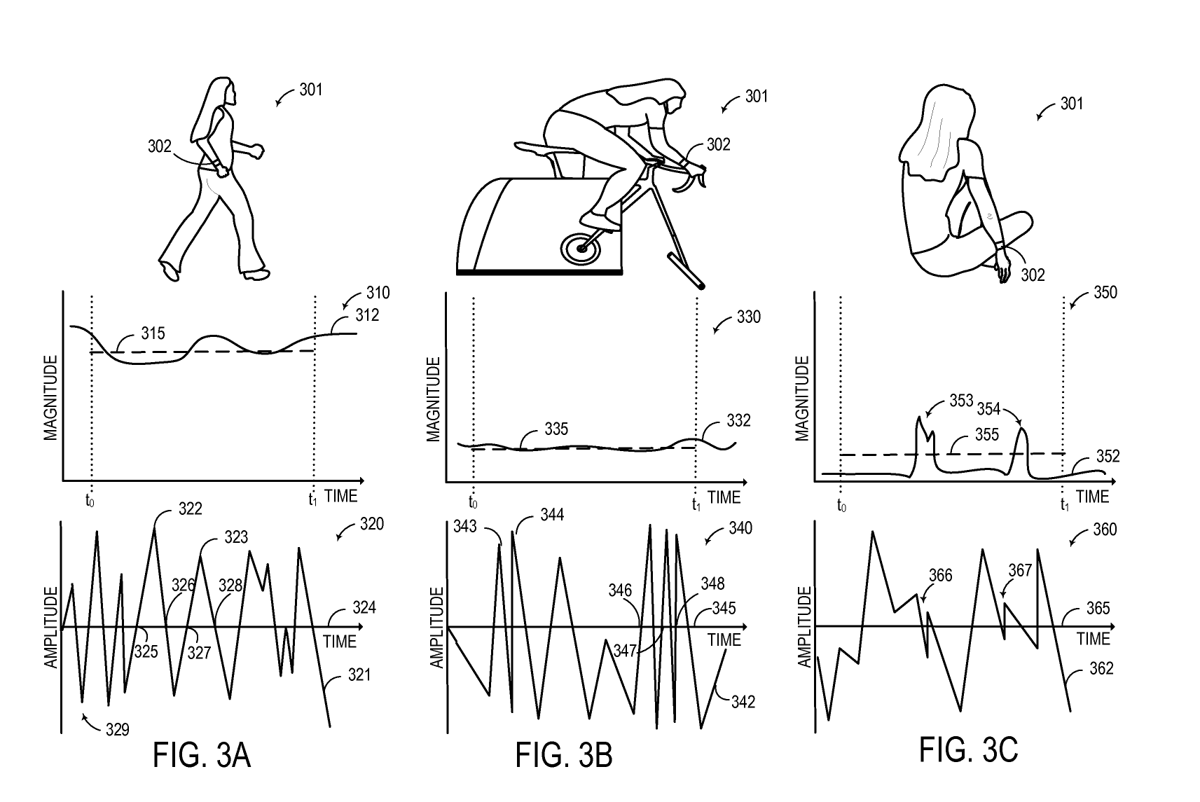

FIGS. 3A-3C show depictions of a user 301 wearing a sensory-and-logic system 302. Sensory-and-logic system 302 is shown in the form of a wearable computing device coupled to the wrist of user 301. Sensory-and-logic system 302 includes a compute system, one or more motion sensors, and an optical heart rate monitor, the optical heart rate monitor comprising an optical source and an optical sensor. FIG. 3A shows user 301 engaged in a state of relatively high movement intensity (e.g., jogging). FIG. 3B shows user 301 engaged in a state of relatively moderate movement intensity (e.g., riding a stationary bicycle), albeit a state where sensory-and-logic system 302 is moving at a relatively low intensity. FIG. 3C shows user 301 engaged in a state of relatively low movement intensity (e.g., sitting).

FIG. 3A shows an example chart 310 for relative movement as seen by a motion sensor coupled within sensory-and-logic device 302. Chart 310 includes plot 312, indicating the magnitude of a signal output by the motion sensor to a compute system over time. Plot 312 may represent the motion from one or more sensors (e.g., x-axis accelerometer, y-axis accelerometer, and z-axis accelerometer). While user 301 is jogging, the user's wrist is in a state of consistent, relatively high movement intensity. Line 315 represents an average magnitude of movement for plot 312 between time t₀ and time t₁.

As described with regards to FIG. 2, the quality of a signal output by the optical sensor may degrade with increased motion. FIG. 3A shows an example chart 320 for an optical signal generated by an optical sensor coupled within sensory-and-logic device 302. Chart 320 includes plot 321, indicating the amplitude of a signal output by the optical sensor to the compute system over time. Each heart beat yields an amplitude peak, such as amplitude peaks 322 and 323. The length of time between consecutive peaks may be used to determine a heart rate of the user. As the amplitude of the peaks may change from heartbeat to heartbeat, heart rate may be calculated by other methods, alternatively or in addition to peak detection. For example, a zero-axis 324 may be determined and applied to the data. Each heart beat thus comprises two zero-crossing events, a negative-to-positive zero-crossing event, such as zero-crossing events 325 and 327, and a positive-to-negative zero-crossing event, such as zero-crossing events 326 and 328. As such, the length of time between alternating zero-crossing events, such as zero-crossing events 325 and 327 or 326 and 328, may be used to determine a heart rate.

However, plot 321 has a relatively low signal-to-noise ratio, due to the movement of user 301. Numerous peaks and zero-crossing events shown in plot 321 do not result from the pulse of user 301, and may not be representative of a heartbeat. For example, the high frequency peaks indicated at 329 may result from leaked light or other adverse conditions. Prior to determining a heart rate, the raw optical signal may first be processed and smoothed, in order to compensate for the detected motion. The raw optical signal may be filtered based on the signal received from the motion sensor in order to remove the motion component from the optical signal, thus improving the accuracy of subsequently derived heart rates.

FIG. 3B shows an example chart 330 for relative movement as seen by a motion sensor coupled within sensory-and-logic device 302. Chart 330 includes plot 332, indicating the magnitude of a signal output by the motion sensor to a compute system over time. While user 301 is riding an exercise bicycle, the user's wrist, and thus sensory-and-logic device 302 is in a state of constant, relatively low movement intensity. Line 335 represents an average magnitude of movement for plot 332 between time t₀ and time t₁.

Although the wrist of user 301 has relatively low movement intensity in this example, the continuous motion of the user's lower body, including footfalls, affect the signal output by the optical sensor, independent of the magnitude of motion indicated by the motion sensor. This movement profile may be derived during “hand-hold workouts”, such as stationary bicycling, stair stepping, or other physical activities where the user's lower body is engaged in continuous, high intensity motion while the user's hands are braced against a stationary surface.

FIG. 3B shows an example chart 340 for an optical signal generated by an optical sensor coupled within sensory-and-logic device 302. Chart 340 includes plot 342, indicating the amplitude of a signal output by the optical sensor to the compute system over time. The motion of user 301 may be sufficient to create harmonic frequencies or doublets that may decrease the signal-to-noise ratio of the optical signal. For example, peaks 343 and 344 may comprise a single heartbeat. Similarly, zero-axis 345 may be crossed repeatedly during a single heartbeat, such as for zero-crossing events 346, 347, and 348. Accordingly, motion compensation may be applied to plot 342 prior to heart rate determination. The amount of motion compensation needed to smooth plot 342 may be less than the amount of motion compensation needed to smooth plot 321, or other optical signals associated with a relatively high amount of detected motion.

FIG. 3C shows an example chart 350 for relative movement as seen by a motion sensor coupled within sensory-and-logic device 302. Chart 350 includes plot 352, indicating the magnitude of a signal output by the motion sensor to a compute system over time. While user 301 is at rest, the user's wrist is in a state of very low movement intensity, though sporadic movements may occur, such as the movements shown at 353 and 354. Line 335 represents an average magnitude of movement for plot 332 between time t₀ and time t₁.

As the wrist of user 301 is predominantly engaged in a state of very low movement intensity in this example, it may not be advantageous to apply motion compensation to the optical signal prior to determining the user's heart rate. FIG. 3C shows an example chart 360 for an optical signal generated by an optical sensor coupled within sensory-and-logic device 302. Chart 360 includes plot 362, indicating the amplitude of a signal output by the optical sensor to the compute system over time, with a zero-axis indicated at 365. The sporadic motion shown at 353 and 354 in plot 352 may give rise to minor peaks and zero-crossing events as shown at 366 and 367. Rather than applying motion compensation to the entire optical signal, the peaks and/or zero-crossing events generated during the sporadic movements may simply be discarded when determining the user's heart rate.

A decision to apply or not apply motion cancellation can be made prior to heart rate determination. According to simplistic strategies, a magnitude of a signal from the motion sensor is compared to a threshold. The magnitude may be based on the maximum magnitude of the motion signal over a period of time, or may be based on a mean magnitude of the motion signal over a period of time. However, it is difficult to set the threshold for these approaches in a fashion which results in motion compensation being applied (or not applied) properly. In FIGS. 3B and 3C, the mean magnitudes of the respective motion signal are similar. Further, the maximum magnitude of plot 352 is greater than the maximum magnitude of plot 332. Using thresholding based on the maximum amplitude or mean amplitude of the motion signal may result in motion compensation applied in FIG. 3C, and not applied in FIG. 3B, where the opposite application may be preferable. Not applying motion compensation when needed may result in the heart rate being derived predominantly based on the motion rate. Applying motion compensation when not needed may result in the heartbeat derived portions of the optical signal being filtered out of the signal. Subsequently, the heart rate may be derived predominantly from random noise. Further, an amount of motion compensation may be a function of the amount of movement. Applying aggressive motion compensation when not needed may also filter out data relevant to heart rate derivation. A sensory-and-logic device may be operable in various modes, such as a workout mode and an everyday mode. However, as shown in FIGS. 3A and 3B, a user may engage in physical activities or exercises featuring various degrees of motion intensity. As such, one type of motion cancellation may not be applicable to all activities a user may engage in during a specific operational mode.

Distinguishing a motion signal representative of a user at rest as opposed to a motion signal representative of a user performing a hand-hold workout may be accomplished by determining a minimum amount of movement over a period of time. For example, the minimum magnitude of plot 332 is greater than the minimum magnitude of plot 352, as the user in FIG. 3B is constantly moving, even if the sensory-and-logic device is moving at a relatively low intensity at the user's wrist. In contrast, the user in FIG. 3C is predominantly at rest (e.g., zero or minimal movement) while making some sporadic movements.

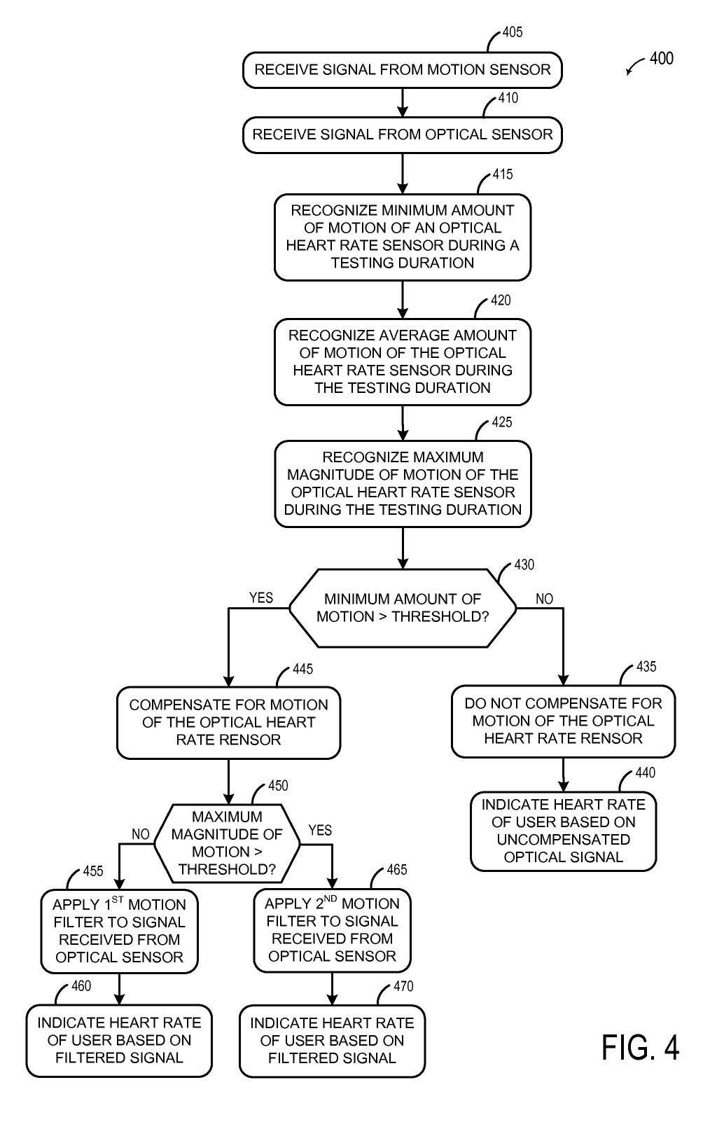

FIG. 4 shows an example method for an optical heart rate sensor in determining whether to apply motion compensation to an optical signal. At 405, method 400 includes receiving a signal from one or more motion sensors. The one or more motion sensors may comprise a gyroscope and/or an accelerometer. The gyroscope and/or accelerometer may be three-axis motion sensors. In those examples, the motion signal may comprise a signal channel for each axis. Continuing at 410, method 400 includes receiving a signal from an optical sensor. The optical signal may indicate reflected illumination from one or more blood vessels illuminated by an optical source through a user's skin.

Continuing at 415, method 400 includes recognizing a minimum amount of motion of the optical heart rate sensor during a testing duration. This may include recognizing a minimum value of the received motion signal during the testing duration. For a motion sensor with multiple signal channels, this may include recognizing minimum values for each signal channel, and then selecting an overall motion minimum out of the minimum values for each signal channel. The testing duration may be a suitable testing duration that comprises two or more heartbeats, for example, eight seconds. The testing duration may be a rolling or moving window, for example, comprising the most recent eight seconds. The testing duration may comprise a plurality of time periods of equal length. For example, an eight second testing duration may comprise eight one-second time periods.

Continuing at 420, method 400 may include recognizing an average amount of motion of the optical heart rate sensor during the testing duration. At 425, method 400 may include recognizing a maximum magnitude of motion of the optical heart rate sensor during the testing duration. The average amount and maximum magnitude of motion may be derived from the received motion signal.

At 430, method 400 includes determining whether the minimum amount of motion during the testing duration, as recognized at 415, is greater than a first predetermined threshold. If the minimum amount of motion is not greater than the predetermined threshold, method 400 may proceed to 435. At 435, method 400 includes not compensating for motion of the optical heart rate sensor. This may include not compensating for motion of the optical heart rate sensor even if the average amount of motion during the testing duration is greater than a second threshold, greater than the first threshold. This may further include not compensating for motion of the optical heart rate sensor even if the maximum magnitude of the motion signal during the testing duration is greater than a third threshold, greater than the first threshold. Continuing at 440, method 400 includes indicating a heart rate of the user based on the uncompensated optical signal.

Returning to 430, if the minimum amount of motion during the testing duration is greater than the first predetermined threshold, method 400 may proceed to 445. At 445, method 400 includes compensating for the motion of the optical heart rate sensor. This may include compensating for motion of the optical heart rate sensor even if the average amount of motion during the testing duration is less than the second threshold, and may further include compensating for the motion of the optical heart rate sensor even if the maximum magnitude of the motion signal during the testing duration is less than the third threshold.

Continuing at 450, method 400 includes determining whether the maximum magnitude of the motion signal during the testing duration is greater than the third threshold. If the maximum magnitude of the motion signal is not greater than the third threshold, method 400 may proceed to 455. At 455, method 400 includes applying a first motion filter to the optical signal based on the motion signal. Continuing at 460, method 400 includes indicating a heart rate of the user based on the filtered optical signal.

Returning to 450, if the maximum magnitude of the motion signal is not greater than the third threshold, method 400 may proceed to 465. At 465, method 400 includes applying a second motion filter to the optical signal based on the motion signal, the second motion filter altering the optical signal more than the first motion filter. Continuing at 460, method 400 includes indicating a heart rate of the user based on the filtered optical signal.

Additionally or alternatively, if the minimum amount of motion during the testing duration exceeds the first predetermined threshold, the second motion filter may be applied to the optical signal if the average amount of motion during the testing duration exceeds the second threshold, while the first motion filter may be applied to the optical signal if the average amount of motion during the testing duration is less than the second threshold.

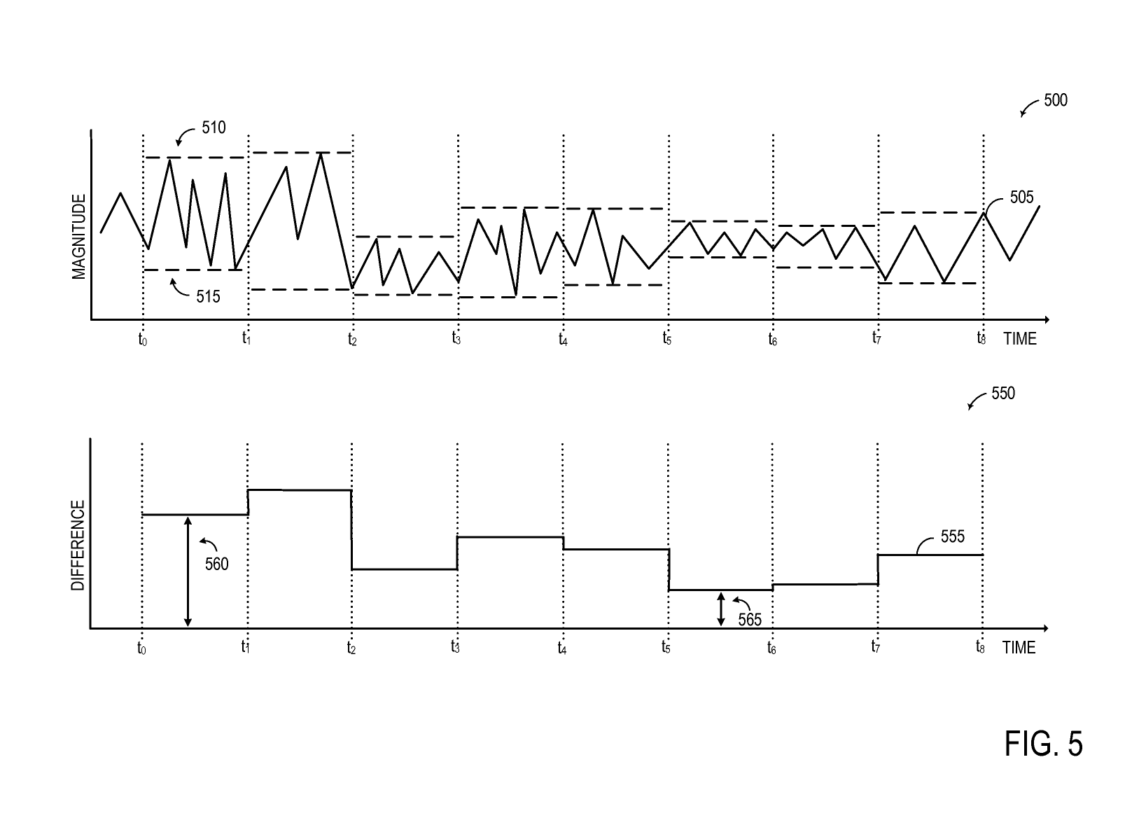

FIG. 5 shows example data traces output by a motion sensor before and after signal processing. FIG. 5 shows chart 500, indicating a magnitude of motion over time. Chart 500 includes plot 505, indicating a raw signal output by a motion sensor over time. Chart 500 indicates a testing duration comprising a plurality of time periods of equal length. In this example, the testing duration runs from time to t₀ time t₈, and comprises eight time periods of equal length. To determine the minimum amount of motion over the testing duration, a difference of the motion signal may be determined for each time period. The difference may be based on the minimum value for the motion signal for that time period and the maximum value of the motion signal for that time period. For example, between time t₀ and time t₁, the maximum value for the motion signal is indicated at 510, and the minimum value for the motion signal is indicated at 515. The difference for this time period may be determined by subtracting the minimum value from the maximum value. A difference may be determined for each time period in this way. The minimum amount of motion may be determined as a difference of the motion signal as opposed to an absolute minimum, as the absolute minimum may not necessarily be equal to zero. For example, even when stationary, an accelerometer may have a non-zero value on at least one axis due to gravity. By determining the difference of the motion signal, background acceleration can be removed from the signal.

FIG. 5 also shows chart 550, indicating the magnitude of the difference for each time period over the testing duration. Chart 550 includes plot 555, indicating a determined difference for each corresponding time period from chart 500. For example, the determined difference for the time period between time t₀ and time t₁ is shown at 560. Determining a minimum value of the motion signal over the testing duration may comprise determining a minimum difference for the motion signal for each of the plurality of time periods. In this example, the minimum difference is indicated at 565, between time t₅ and time t₆. This difference represents the minimum amount of motion over the testing duration and may be compared to a predetermined threshold to determine whether motion compensation should be applied to an optical signal during the testing duration.

As described with regard to FIG. 4, the motion sensor may be a three-axis motion sensor, such as an accelerometer. In such examples, plot 505 may represent one signal channel. Minimum differences may be determined for each signal channel based on the differences of each signal channel for each of the time periods. An overall motion minimum may then be determined based on the determined minimum difference for each signal channel. The overall motion minimum may then be compared to a threshold.

For a testing duration (T) comprising a plurality of time periods (t), the difference for each signal channel may be determined for each time period based on the following equations:

X_diff(t)=Max(X(t))−Min(X(t))

Y_diff(t)=Max(Y(t))−Min(Y(t))

Z_diff(t)=Max(Z(t))−Min(Z(t))

The minimum difference for each signal channel during the testing duration may then be determined based on the following equations:

X_{—}M(T,t)=Min(X_{—}diff(t), X_diff(t−1), . . . X_{—}diff(t−T+1))

Y_{—}M(T,t)=Min(Y_{—}diff(t), Y_{—}diff(t−1), . . . Y_diff(t−T+1))

Z_{—}M(T,t)=Min(Z_{—}diff(t), Z_{—}diff(t−1), . . . Z_diff(t−T+1))

A minimum motion value may then be determined based on the minimum differences for each signal channel.

V(t)=Min(X_{—}M(T,t),Y_{—}M(T,t),Z_{—}M(T,t))

V(t) may then be compared to a threshold to determine whether or not to apply motion cancellation to the signal prior to determining a heart rate.

As evident from the foregoing description, the methods and processes described herein may be tied to a sensory-and-logic system of one or more machines. Such methods and processes may be implemented as a computer-application program or service, an application-programming interface (API), a library, firmware, and/or other computer-program product. FIGS. 1A and 1B show one, non-limiting example of a sensory-and-logic system to enact the methods and processes described herein. However, these methods and process may also be enacted on sensory-and-logic systems of other configurations and form factors, as shown schematically in FIG. 6.

FIG. 6 schematically shows a form-agnostic sensory-and-logic system 610 that includes a sensor suite 612 operatively coupled to a compute system 614. The compute system includes a logic machine 616 and a data-storage machine 618. The compute system is operatively coupled to a display subsystem 620, a communication subsystem 622, an input subsystem 624, and/or other components not shown in FIG. 6.

Logic machine 616 includes one or more physical devices configured to execute instructions. The logic machine may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

Logic machine 616 may include one or more processors configured to execute software instructions. Additionally or alternatively, the logic machine may include one or more hardware or firmware logic machines configured to execute hardware or firmware instructions. Processors of the logic machine may be single-core or multi-core, and the instructions executed thereon may be configured for sequential, parallel, and/or distributed processing. Individual components of a logic machine optionally may be distributed among two or more separate devices, which may be remotely located and/or configured for coordinated processing. Aspects of a logic machine may be virtualized and executed by remotely accessible, networked computing devices in a cloud-computing configuration.

Data-storage machine 618 includes one or more physical devices configured to hold instructions executable by logic machine 616 to implement the methods and processes described herein. When such methods and processes are implemented, the state of the data-storage machine may be transformed—e.g., to hold different data. The data-storage machine may include removable and/or built-in devices; it may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. The data-storage machine may include volatile, nonvolatile, dynamic, static, read/write, read-only, random-access, sequential-access, location-addressable, file-addressable, and/or content-addressable devices.

Data-storage machine 618 includes one or more physical devices. However, aspects of the instructions described herein alternatively may be propagated by a communication medium (e.g., an electromagnetic signal, an optical signal, etc.) that is not held by a physical device for a finite duration.

Aspects of logic machine 616 and data-storage machine 618 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

Display subsystem 620 may be used to present a visual representation of data held by data-storage machine 618. This visual representation may take the form of a graphical user interface (GUI). As the herein described methods and processes change the data held by the storage machine, and thus transform the state of the storage machine, the state of display subsystem 620 may likewise be transformed to visually represent changes in the underlying data. Display subsystem 620 may include one or more display subsystem devices utilizing virtually any type of technology. Such display subsystem devices may be combined with logic machine 616 and/or data-storage machine 618 in a shared enclosure, or such display subsystem devices may be peripheral display subsystem devices. Display 20 of FIGS. 1A and 1B is an example of display subsystem 620.

Communication subsystem 622 may be configured to communicatively couple compute system 614 to one or more other computing devices. The communication subsystem may include wired and/or wireless communication devices compatible with one or more different communication protocols. As non-limiting examples, the communication subsystem may be configured for communication via a wireless telephone network, a local- or wide-area network, and/or the Internet. Communication suite 24 of FIGS. 1A and 1B is an example of communication subsystem 622.

Input subsystem 624 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity. Touch-screen sensor 32 and push buttons 34 of FIGS. 1A and 1B are examples of input subsystem 624.

Sensor suite 612 may include one or more different sensors—e.g., a touch-screen sensor, push-button sensor, microphone, visible-light sensor, ultraviolet sensor, ambient-temperature sensor, contact sensors, and/or GPS receiver—as described above with reference to FIGS. 1A and 1B. Sensor suite 612 may include motion sensor suite 626. Motion sensor suite 626 may include one or more of an accelerometer, gyroscope, magnetometer, or other suitable motion detectors. Sensor suite 612 may further include optical heart rate sensor 628. As described herein, optical heart rate sensor 628 may include optical source 630 and optical sensor 632. Compute system 614 may include optical heart rate control subsystem 634, which may be communicatively coupled to logic machine 616 and data-storage machine 618. Optical source 630 may comprise one or more LED emitters, for example, while optical sensor 632 may comprise one or more photodiodes matched to detect light at frequencies that are based on the frequencies of light output by the optical source. Optical source 630 may be configured to illuminate one or more blood vessels 650 through the skin 652 of the user, and optical sensor 632 may be configured to measure illumination reflected from or transmitted through blood vessels 650.

Optical heart rate control subsystem 634 may receive raw signals from optical sensor 632, and may further process the raw signals to determine heart rate, caloric expenditures, etc. Processed signals may be stored and output via compute system 614. Control signals sent to optical source 630 and optical sensor 632 may be based on signals received from optical sensor 632, signals derived from sensor suite 612, information stored in data-storage machine 618, input received from communication subsystem 622, input received from input subsystem 624, etc.

The configurations and approaches described herein are exemplary in nature, and that these specific implementations or examples are not to be taken in a limiting sense, because numerous variations are feasible. The specific routines or methods described herein may represent one or more processing strategies. As such, various acts shown or described may be performed in the sequence shown or described, in other sequences, in parallel, or omitted.

The subject matter of this disclosure includes all novel and non-obvious combinations and sub-combinations of the various processes, systems and configurations, and other features, functions, acts, and/or properties disclosed herein, as well as any and all equivalents thereof.

1. A method of optical heart rate sensing, comprising:
recognizing a minimum amount of motion of an optical heart rate sensor during a testing duration;
recognizing an average amount of motion of the optical heart rate sensor during the testing duration;
applying compensation for motion of the optical heart rate sensor if the minimum amount of motion during the testing duration exceeds a first threshold, even if the average amount of motion during the testing duration is less than a second threshold, greater than the first threshold; and
omitting compensation for motion of the optical heart rate sensor if the minimum amount of motion during the testing duration is below the first threshold, even if the average amount of motion during the testing duration is greater than the second threshold.

2. The method of claim 1, where recognizing the minimum amount of motion of the optical heart rate sensor during the testing duration further comprises:
receiving a motion signal from one or more motion sensors; and
recognizing a minimum value of the motion signal during the testing duration.

3. The method of claim 2, where the testing duration comprises a plurality of time periods of equal length, and where recognizing a minimum value of the motion signal during the testing duration further comprises:
for each of the plurality of time periods, determining a difference of the motion signal based on a minimum value of the motion signal for that time period and a maximum value of the motion signal for that time period.

4. The method of claim 3, where recognizing a minimum value of the motion signal during the testing duration further comprises:
determining a minimum difference for the testing duration based on the determined differences for the motion signal for each of the plurality of time periods.

5. The method of claim 4, where the one or more motion sensors comprise a three-axis accelerometer, and where the motion signal comprises a signal channel for each axis.

6. The method of claim 5, where determining the minimum difference for the testing duration based on the determined differences for each of the plurality of time periods further comprises:
for each time period, determining a difference for each signal channel based on a minimum value of the signal channel for that time period and a maximum value of the signal channel for that time period; and
determining minimum differences for each signal channel over the testing duration based on the determined differences for each signal channel for each of the plurality of time periods.

7. The method of claim 6, where determining the minimum difference for the testing duration based on the determined differences for each of the plurality of time periods further comprises:
determining an overall motion minimum based on the determined minimum differences for each signal channel; and
comparing the overall motion minimum to the first threshold.

8. The method of claim 1, where compensating for motion of the optical heart rate sensor further comprises:
applying a first motion filter to a signal received from one or more optical sensors based on motion of the optical heart rate sensor; and
indicating a heart rate of a user based on the filtered signal.

9. The method of claim 8, further comprising:
applying a second motion filter to the signal received from the one or more optical sensors if the minimum amount of motion during the testing duration exceeds the first threshold and the average amount of motion during the testing duration exceeds the second threshold, the second motion filter altering the signal received from the one or more optical sensors more than the first motion filter; and
indicating a heart rate of a user based on the filtered signal.

10. A method of optical heart rate sensing, comprising:
receiving an optical signal from one or more optical sensors;
receiving a motion signal from one or more motion sensors;
determining a minimum difference for the motion signal over a testing duration;
determining a maximum magnitude for the motion signal over the testing duration;
applying a first motion filter to the optical signal during conditions where the minimum difference for the motion signal over the testing duration is greater than a first threshold and the maximum magnitude for the motion signal over the testing duration is less than a second threshold, the second threshold greater than the first threshold; and
not applying a motion filter to the optical signal during conditions where the minimum difference for the motion signal over the testing duration is less than the first threshold even if the maximum magnitude for the motion signal over the testing duration is greater than the second threshold.

11. The method of claim 10, where the optical signal indicates reflected illumination from one or more blood vessels illuminated by an optical source through a user's skin.

12. The method of claim 11, further comprising:
indicating a heart rate of a user based on the filtered optical signal during conditions where the minimum difference for the motion signal over the testing duration is greater than the first threshold and the maximum magnitude for the motion signal over the testing duration is less than the second threshold; and
indicating a heart rate of the user based on the unfiltered optical signal during conditions where the minimum difference for the motion signal over the testing duration is less than the first threshold even if the maximum magnitude for the motion signal over the testing duration is greater than the second threshold.

13. The method of claim 10, where the testing duration comprises a plurality of time periods of equal length, and where determining a minimum difference of the motion signal during the testing duration further comprises:
for each of the plurality of time periods, determining a difference of the motion signal based on a minimum value of the motion signal for that time period and a maximum value of the motion signal for that time period; and
determining a minimum difference for the testing duration based on the determined differences of the motion signal for each of the plurality of time periods.

14. The method of claim 13, where the one or more motion sensors comprise a three-axis accelerometer, and where the motion signal comprises a signal channel for each axis.

15. The method of claim 14, where determining the minimum difference for the motion signal over the testing duration further comprises:
for each of the plurality of time periods, determining a difference for each signal channel based on a minimum value of the signal channel for that time period and a maximum value of the signal channel for that time period; and
determining minimum differences for each signal channel over the testing duration based on the determined differences for each signal channel for each of the plurality of time periods.

16. The method of claim 15, where determining the minimum difference for the motion signal over the testing duration further comprises:
determining an overall motion minimum based on the determined minimum difference for each signal channel; and
comparing the overall motion minimum to the first threshold.

17. The method of claim 10, further comprising:
applying a second motion filter to the optical signal during conditions where the minimum difference for the motion signal is greater than the first threshold and the maximum magnitude for the motion signal is greater than the second threshold, the second motion filter adjusting the optical signal more than the first motion filter.

18. An optical heart rate sensor, comprising:
an optical source configured to illuminate one or more blood vessels through a user's skin,
an optical sensor configured to measure illumination from the one or more blood vessels;
a three-axis accelerometer; and
a storage machine holding instructions executable by a logic machine to:
receive an optical signal from the optical sensor;
receive a motion signal from the three-axis accelerometer, the motion signal comprising three signal channels;
determine a minimum difference for the motion signal during a testing duration;
determine a maximum magnitude for the motion signal during the testing duration;
apply a first motion filter to the optical signal during conditions where the minimum difference for the motion signal over the testing duration is greater than a first threshold and the maximum magnitude for the motion signal over the testing duration is less than a second threshold, the second threshold greater than the first threshold;
not apply a motion filter to the optical signal during conditions where the minimum difference for the motion signal over the testing duration is less than the first threshold even if the maximum magnitude for the motion signal over the testing duration is greater than the second threshold;
indicate a heart rate of a user based on the filtered optical signal during conditions where the minimum difference for the motion signal over the testing duration is greater than the first threshold and the maximum magnitude for the motion signal over the testing duration is less than the second threshold; and
indicate a heart rate of the user based on the unfiltered optical signal during conditions where the minimum difference for the motion signal over the testing duration is less than the first threshold even if the maximum magnitude for the motion signal over the testing duration is greater than the second threshold.

19. The optical heart rate sensor of claim 18, where the instructions to determine the minimum difference for the motion signal over the testing duration, comprise instructions executable to:
for each of the plurality of time periods, determine a difference for each signal channel based on a minimum value of the signal channel for that time period and a maximum value of the signal channel for that time period; and
determine minimum differences for each signal channel over the testing duration based on the determined differences for each signal channel for each of the plurality of time periods.

20. The optical heart rate sensor of claim 19, where the instructions to determine the minimum difference for the motion signal over the testing duration, comprise instructions executable to:
determine an overall motion minimum based on the determined minimum difference for each signal channel; and
compare the overall motion minimum to the first threshold.
